(19) United States
(12) Patent Application Publication
(10) Pub. No.: US 2023/0123043 A1
(43) Pub. Date: Apr. 20, 2023

(54) DEVICE TO GUIDE INCISIONS IN PENILE SURGERY

(71) Applicant: ALEXANDRE DE FREITAS MIRANDA, Rio de Janeiro (BR)

(72) Inventor: ALEXANDRE DE FREITAS MIRANDA, Rio de Janeiro (BR)

(21) Appl. No.: 17/943,833

(22) Filed: Sep. 13, 2022

(30) Foreign Application Priority Data
Oct. 15, 2021 (BR) 102021020776-0

(51) Int. Cl. A61B 17/3209

(52) U.S. Cl. CPC A61B 17/3209; A61B 2017/320052

(57) ABSTRACT

The present invention relates to a device to guide incisions in penile surgery comprising an auxetic EVA sheet (1) that is fixed in the penile corpora cavernosa, said device having star-shaped perforations (2) that create a triangle rotated system, which allows to make specific micro incisions in the penile corpora cavernosa, generating simultaneous two-dimensional expansion of the tunica albuginea, providing a real increase in length and diameter (girth), in addition to correcting any other type of penile deformity, like penile curvature.

### FIELD OF THE INVENTION

The present invention relates to a device to guide incisions in penile surgery comprising an EVA sheet with auxetic property that is fixed in the penile corpora cavernosa, said device having star-shaped perforations that create a system of triangles, which allows to make specific micro incisions in the penile corpora cavernosa, generating simultaneous two-dimensional expansion of the tunica albuginea, providing a real increase in length and diameter (girth), and correcting any other type of penile deformity, like penile curvature.

### BACKGROUND OF THE INVENTION

Some medical conditions can cause erectile dysfunction with penile length and girth loss, such as Peyronie's disease (PD), postradical prostatectomy, erectile dysfunction following ischemic priapism, among others.

Some surgical and non-surgical treatments have been created to solve the problem of penile volume loss.

Non-surgical treatments involve the use of apparatuses and/or devices of external mechanical traction, vacuum, electromagnetic stimulation and weight, among others, which are fixed externally to the penis.

Among the surgical treatments, there is the penile prosthesis implantation including multiple transverse tunica albuginea incisions without graft, which straightens and lengthens the penis.

Another surgical treatment for penile curvature is the penile prosthesis implantation with a graft, usually of bovine pericardium, increasing the short side of the penile curvature and straightening the penis. However, the result is limited to curvature correction without an increase in penile length or girth.

Some techniques generate penile length gain without a graft, based on the “sliding” technique.

Another surgical solution is the so-called “mesh” technique, which consists of multiple small transversal incisions made in the corpora cavernosa, correcting penile curvature and restoring the length loss but reducing the girth.

In this context, after medical studies, it was observed that the direction of micro incisions determines the expansion axis of the corpora cavernosa. Thus, transverse incisions increase the penis length and longitudinal incision increase the penis girth.

Hence, it is not possible to increase both length and girth at the same place on the corpora cavernosa, a phenomenon that occurs due to mechanical properties of the tunica albuginea.

In order to overcome the problems mentioned, the state of the art teaches some methods, devices and apparatuses to correct erectile dysfunction, and patent documents BR102014028853-8 and BR20202021223-6 refer to devices that are externally fixed to the penis, to correct penile curvatures and maintain the erect penis.

The devices described in the documents above are intended to pull the penis, without being able to effectively increase the penis length or girth. In addition, all devices and/or apparatuses attached externally to the penis are extremely uncomfortable for the user, and since they are applied to a delicate and sensitive organ, they can even injure the penis.

Another state of the art document is U.S. Pat. No. 5,868,729, that discloses a surgical tool that includes a malleable elongated body having a flexible plunger rod mounted slidingly into an orifice and having a conical and rounded front end to facilitate insertion into the corpora cavernosa. A needle with fixed suture material can be placed at the end of the rod, which can be retracted to the elongated body orifice. In practice, the tool can be inserted into a body structure, such as one of the cavernous bodies of a penis. The outer surface of the tool body can be equipped with measure markings that allow the user to measure the depth at which the cylinder was inserted into a body structure. After the tool has been malleably bent to access the cavernous bodies as intended, the flexible plunger rod, with the fixed threaded suture needle, is slid along the hole to extend the needle out of its rounded and conical end. The needle is manually pulled out of the body structure. After the tool is removed, the suture material remains threaded into the body structure.

The tool and technique described in the document above present the inconvenience of serving only to dilate the corpora cavernosa, without altering its dimensions.

Another state of the art document is WO03/103537, which discloses a surgically implanted penile prosthesis, including an apical prosthetic element to be implanted as a cushion in a subglandar position, between the distal surface of the cavernous bodies and the glans, such as an apical prosthetic element with adequate size and shape to result, after implantation, in a general increase in the glans volume without substantial changes in the typical anatomical shape of the glans itself. The prosthesis can be associated with a pericavernous prosthesis composed of two pericavernous prosthetic elements to be placed laterally, face to face, outside the cavernous bodies along the entire penile length, the two pericavernous prosthetic elements being essentially semicylindrical or having the form of a cylindrical segment of reduced height or having a crescent-shaped cross-section.

The technique described in the document above provides only the increase of the glans and does not achieve the goal of simultaneously increasing the penis length and girth. In addition, the intervention may result in glans necrosis, hematoma or infection, requiring removal of the implant.

Next, we present a simplified summary of the embodiments described in the present invention, and such summary is not an extensive overview of all the embodiments contemplated herein. This summary does not intend to identify fundamental or critical elements, nor to outline the scope of such embodiments. Its sole purpose is to present some embodiment concepts described in simplified form, such as an introduction to the more detailed description that will be presented below.

### SUMMARY OF THE INVENTION

The present invention relates to a device to guide incisions in penile surgery comprising an EVA sheet with an auxetic property that is fixed in the corpora cavernosa, said auxetic template having star-shaped perforations. These perforations guide the electric scalpel to generate micro incisions with the same star shape of the auxetic template in the corpora cavernosa. The star shape incision creates triangles rotated system that allows the simultaneous two-dimensional expansion of the tunica albuginea, providing a real increase in length and diameter (girth) and correcting any other type of deformity of the organ, like, penile curvature.

The main advantage of the present invention is the provision of a homogeneous application of star-shaped micro incisions in the corpora cavernosa which, in addition to solving any penis deformity, generates the simultaneous two-dimensional expansion of the tunica albuginea.

Another advantage of the present invention is the possibility of creating sufficient space for penile prosthesis implantation in cases of corpora cavernosa fibrosis, such as after priapism or explantation of infected penile prosthesis.

Additional advantage is that the auxetic template can be used in plastic surgery, when there is a need for tissue expansion, such as in cases of large burns or difficult abdominal closures.

For achieving the previous and related objectives, one or more embodiments comprise the aspects that will be described below and are specifically defined in the claims. The following description and the attached drawings provide certain illustrative details of the aspects of the described embodiments. However, these aspects indicate only some of the various ways in which the principles of various embodiments can be used. In addition, it is intended that the described embodiments include these aspects and their equivalents.

### BRIEF DESCRIPTION OF THE FIGURES

The characteristics, nature and advantages of this description will be more evident from the detailed description set out below when read in conjunction with the drawings, in which the same references refer to the same elements.

FIG. 1—illustrates the auxetic template, object of the present invention;

FIG. 2A—illustrates an enlarged view detailing the star-shaped perforation of the auxetic template;

FIG. 2B—illustrates an enlarged view detailing the final defect caused by the star-shaped perforation of the auxetic template;

FIG. 3—illustrates a detailed schematic view of the auxetic behavior created by rotating the equilateral triangles that make up the auxetic template;

FIG. 4A—illustrates a view of the circumferential application of the auxetic template in the corpora cavernosa of a penis to increase the penile length and girth;

FIG. 4B—illustrates a penis view of FIG. 4A after the micro incisions in the corpora cavernosa;

FIG. 5A—illustrates a view of the unilateral application of the auxetic template in the penis corpora cavernosa to correct dorsal curvature;

FIG. 5B—illustrates a penis view of FIG. 5A after making the micro incisions in the corpora cavernosa and its correction;

FIG. 6A—illustrates a lateral view of the unilateral application of the auxetic template in the penis corpora cavernosa to correct hourglass deformity and dorsal curvature;

FIG. 6B—illustrates a penis view of FIG. 6A after making the micro incisions in the corpora cavernosa and the correction of the deformities;

FIG. 7A—illustrates a view of the unilateral application of the auxetic template in the penis corpora cavernosa to correct a lateral curvature;

FIG. 7B—illustrates a penis view of FIG. 7A after making the micro incisions in the corpora cavernosa and correct the deformitie.

### DETAILED DESCRIPTION OF THE INVENTION

In accordance with what the attached figures show, the present invention refers to a DEVICE TO GUIDE INCISIONS IN PENILE SURGERY comprising an EVA sheet (1) with auxetic property that is fixed in the penile corpora cavernosa with surgical thread stitches. The device allows guiding the surgeon to make star-shaped, auxetic micro incisions, aligned at specific points of the penile corpora cavernosa, generating simultaneous two-dimensional expansion of the tunica albuginea, providing a real increase in length and diameter (girth) and correcting any other type of penile deformity, such as penile curvature. After surgery, the auxetic EVA sheet (1) is removed from the corpora cavernosa.

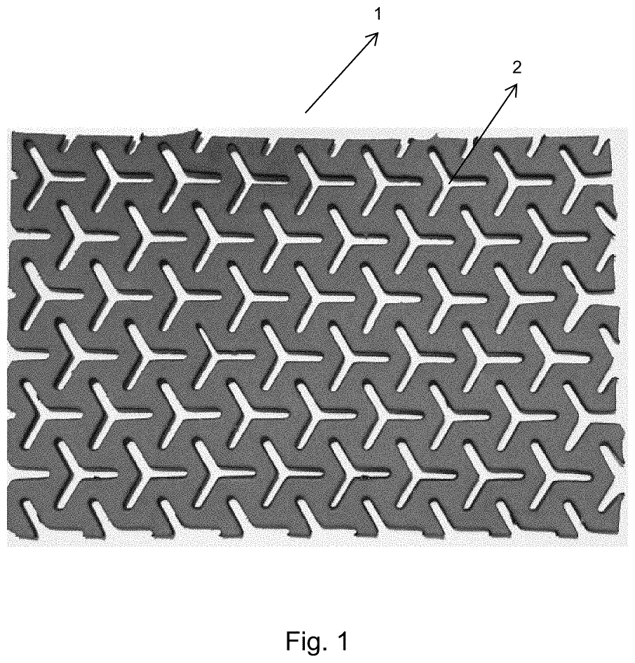

Now we refer to FIG. 1, which illustrates the device that consists of an auxetic EVA sheet (1) with 1 mm thickness, with a size ranging from 12 cm×8 cm to 12 cm×15 cm, with a plurality of star-shaped perforations (2) equidistant from each other, which create a triangle rotated system that allows simultaneous longitudinal and transversal expansion. The microincisions made through these perforations create the same system of rotating triangles in the corpora cavernosa.

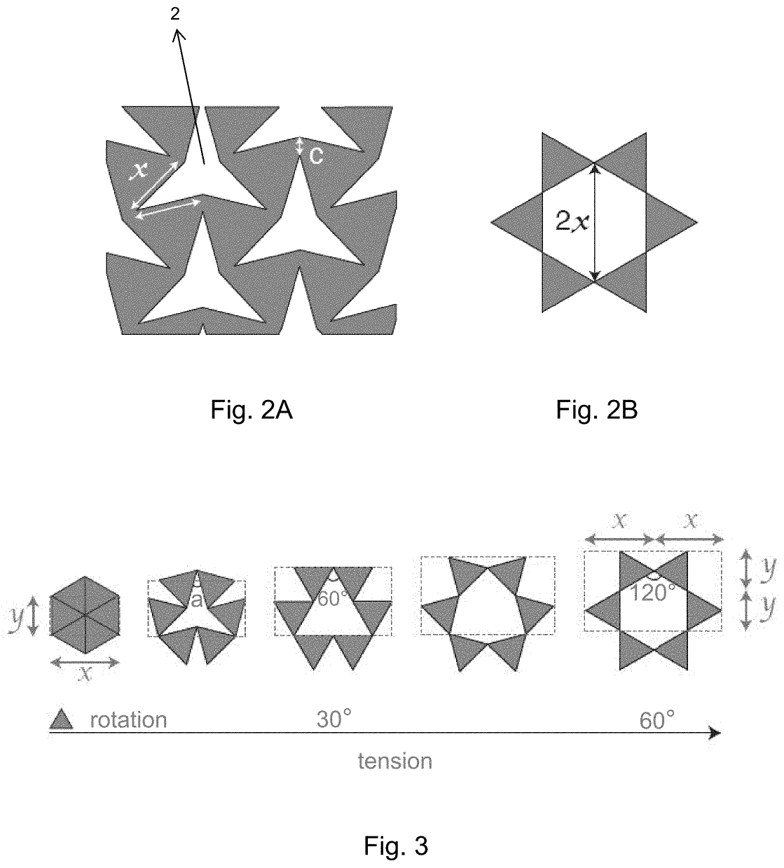

Now we refer to FIGS. 2A and 2B, which illustrate two factors that contribute to the tissue expansiveness and final size of the defect. In FIG. 2A some traction and, in FIG. 2B, the maximum traction can be observed in the tissue. If the incision size, that is, the star-shaped (X) cut (2) is very large, we may have a defect that needs to be closed by placing a graft and, if the size is too small, the rotation capacity of the triangles is reduced, thus decreasing the final area of the tissue expansion.

In addition, the degree of connection between the triangles (C) is another factor that alters the final result of tissue expansion and resistance to tension. If this value is too small, the tissue tears in (C), and if this value is high, it will be difficult to rotate the triangle and increase the tissue area.

Taking these factors into account, the value of (X) ranges from 4 mm to 9 mm, and the connection(C) ranges from 2 mm to 6 mm.

Now we refer to FIG. 3, which illustrates the behavior by rotating the equilateral triangles on the x and y axes of the auxetic EVA sheet (1), wherein the traction exerted by the EVA sheet (1) increases the angle (a) between the triangles, producing an open structure and, thus, a negative Poisson's Rate(PR) in the tunica albuginea. The PR is a mechanical property of the materials and is defined as the percentage reduction of the girth of the stretched material, divided by the percentage of gain in length.

Thus, through the star-shaped micro incisions obtained by the auxetic EVA sheet (1), it is possible to obtain a negative PR in the tunica albuginea and, consequently, a simultaneous two-dimensional expansion of the tissue, providing a real increase in length and girth (girth) and correcting any type of penile deformity, like penile curvature.

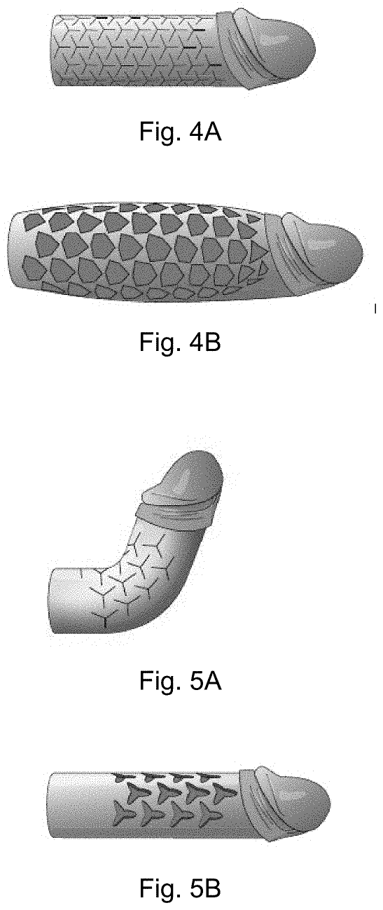

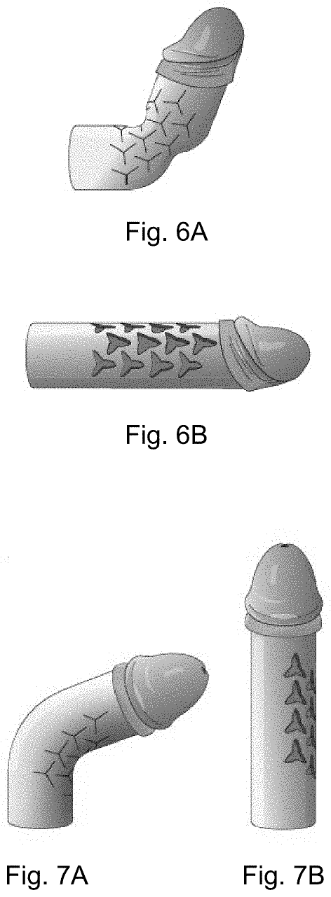

Now we refer to the other FIGS. 4A, 4B, 5A, 5B, 6A, 6B, 7A and 7B, which illustrate examples of penis, before and after, respectively, of the application of auxetic incisions in the corpora cavernosa, to correct penile deformities.

Those skilled in the art will easily understand that modifications can be made in the invention without departing from the concepts exposed in the description above. These modifications should be considered as included within the scope of the invention. Consequently, the particular embodiments described in detail above are merely illustrative and do not limit the scope of the invention, to which should be given the full extent of the attached claims and any and all equivalents thereof.

1. Device to guide incisions in penile surgery, said device being fixed in the penile corpora cavernosa with surgical thread stitches, and removed after corpora cavernosa microincisions, CHARACTERIZED in that it is constituted by an auxetic EVA sheet (1) having 1 mm thickness, with a size ranging from 12 cm×8 cm to 12 cm to 15 cm, with a plurality of star-shaped perforations (2) equidistant from each other, which create a triangle rotated system that allows simultaneous longitudinal and transversal expansion. The microincisions made through these perforations create the same system of rotating triangles in the corpora cavernosa, wherein, in the star-shaped holes (2) the value of (X) varies from 4 mm to 9 mm, and the value of the connection between the triangles (C) varies from 2 mm to 6 mm.

2. Device to guide incisions in penile surgery, according to claim 1, CHARACTERIZED in that the star-shaped micro incisions aligned in the corpora cavernosa cause simultaneous two-dimensional expansion of the tunica albuginea.

3. Device to guide incisions in penile surgery, according to claim 1, CHARACTERIZED in that the star-shaped micro incisions aligned in the corpora cavernosa cause correction of the penis uniplanar, biplanar and multiplanar curvatures, and the hourglass deformity.
